(19) United States
(12) Patent Application Publication
(10) Pub. No.: US 2022/0339197 A1
(43) Pub. Date: Oct. 27, 2022

(54) USE OF HEMATOPOIETIC STEM CELLS IN PREPARATION OF FORMULATION FOR TREATING VIRAL DISEASES

(71) Applicant: GZ CEDICINE BIOTECH CO., LTD., Guangzhou (CN)

(72) Inventors: Qingyan ZOU, Guangzhou (CN); Shiyue LI, Guangzhou (CN); Bin WANG, Guangzhou (CN); Haixia LIU, Guangzhou (CN); Shaoyin DU, Guangzhou (CN); Haiyan LIANG, Guangzhou (CN); Xianfeng DING, Guangzhou (CN); Zhijun WU, Guangzhou (CN); Shaobin DUAN, Guangzhou (CN)

(21) Appl. No.: 17/766,071

(22) PCT Filed: Oct. 14, 2020
(86) PCT No.: PCT/CN2020/120769

§ 371 Date: Apr. 1, 2022

(30) Foreign Application Priority Data
Oct. 14, 2019 (CN) 201910973691.6

(51) Int. Cl. A61K 35/28; C12N 5/0789; A61P 31/20; A61P 31/16; A61P 31/22

(52) U.S. Cl. CPC A61K 35/28; C12N 5/0647; A61P 31/20; A61P 31/16; A61P 31/22

(57) ABSTRACT

The present invention relates to a new use for hematopoietic stein cells, and especially to the use of hematopoietic stein cells in the preparation of a formulation for treating viral diseases. The viral diseases are selected from viral hepatitis, influenza, viral interstitial pneumonia, viral encephalitis and avian influenza. The new use of hematopoietic stein cells provided by the present invention may have a good application prospect for the treatment of influenza viral diseases.

### FIELD OF INVENTION

The present invention relates to a new use for hematopoietic stem cells, and especially to the use of hematopoietic stem cells in the preparation of a formulation for the treatment of viral diseases.

### BACKGROUND OF INVENTION

Various diseases caused by virus infection seriously endanger the health and lives of humans and animals. So far, more than 1,200 viruses and tens of thousands of virus subtypes and variants have been found to cause disease in humans all over the world. Since the 1980s, two-thirds of newly discovered epidemic infectious diseases are caused by viral infections. Among them, viral diseases with high incidence and great harm are acute and chronic hepatitis caused by A, B, C, D, E and G hepatitis viruses; retinitis, keratitis, interstitial pneumonia, encephalitis, genital herpes, herpes zoster and herpes labialis caused by 8 human herpes viruses; respiratory virus infections Bronchitis, pneumonia, measles, mumps, and polio; acute gastroenteritis caused by enteroviruses, diarrhea of travelers and infants, etc.; influenza A and B viruses and avian influenza viruses often cause seasonal global or A pandemic in some areas.

Investigators of Cambridge University in March, 2019, the global second example AIDS patient was successfully cleared of HIV, this example patient's name has not been revealed, known as “London Patient” (HIV-1 remission following CCR5Δ32/Δ32 haematopoietic stem-cell transplantation Ravindra K. Gupta, Sultan Abdul-Jawad, Laura E. McCoy, Hoi Ping Mok, Dimitra Peppa, Maria Salgado, Javier Martinez-Picado, Monique Nijhuis, Annemarie M. J. Wensing, Helen Lee, Paul Grant, Eleni Nastouli, Jonathan Lambert, Matthew Pace, Fanny Salasc, Christopher Monit, Andrew J. Innes, Luke Muir, Laura Waters, John Frater, Andrew M. L. Lever, Simon G. Edwards, Ian H. Gabriel & Eduardo Olavarria Nature, volume 568, pages244-248 (2019)). The global first example successfully cures the patient name of AIDS as Timothy Ray Brown, also referred to as “Berlin Patient”, which has cured AIDS in 2007.

The “Berlin Patient” and the new “London Patient” both accept stem cell transplantation therapy from the donor, while the donor carries a rare gene mutation, whose HIV co-receptor CCR5 is homozygous mutation (CCR5 Δ32/Δ32). These patients have resistance to HIV after stem cell transplantation. According to a “natural” article, this “London Patient” has alleviateed up to 18 months after stopping taking the anti-retroviral drug.

The main authors of this study, Radindra Gupta, a researcher of United Kingdom Cambridge University Medical Line, indicates that the “ Berlin Patient ” has been demonstrated to not be an abnormal phenomenon by using a similar method, and the treatment method of this two patients did eliminate HIV.

### SUMMARY OF THE INVENTION

It is a primary object to provide a new use of hematopoietic stem cells, and especially to the use of hematopoietic stem cells in the preparation of a formulation for the treatment of viral diseases, wherein said viral disease is selected from one of the group: viral hepatitis, influenza, viral interstitial pneumonia, viral encephalitis and avian influenza.

According to the further technical feature of the use in the present invention, hematopoietic stem cells are umbilical cord blood mononuclear cells extracted from animal umbilical cord blood, and the proportion of hematopoietic stem cells (CD34 and CD133 cells) should be greater than 1%.

According to the further technical feature of the use in the present invention, hematopoietic stem cells are bone marrow blood mononuclear cells extracted from animal bone marrow blood, and the proportion of hematopoietic stem cells (CD34 and CD133 cells) should be greater than 1%.

According to the further technical feature of the use in the present invention, hematopoietic stem cells are single core cells in tissue blood extracted from young animal tissues, and the proportion of hematopoietic stem cells (CD34 and CD133 cells) should be greater than 1%.

Research results of the present invention show that stem cell therapy may be effective for viral diseases. The new use of hematopoietic stem cells provided by the present invention may have a good application prospect for the treatment of influenza viral diseases.

### DETAIL DESCRIPTION OF THE INVENTION

Definitions: When the present invention is specifically described, it is necessary to define and interpret certain terms to be used herein to facilitate understanding of the present invention.

Hematopoietic stem cells: hematopoietic stem cells (HSC) refer to stem cells in bone marrow, have self-renewal capacity and can differentiate into various blood cell precursor cells, and finally generate various blood cell components, including red blood cells, leukocytes and platelets, and they can also be differentiated into various other cells. They have good differentiation and proliferation ability, and stem cells can rescue many people suffering from blood diseases, most commonly leukemia. Donation of hematopoietic stem cells has no significant harm to the donor's body. At present, there are mainly four sources of hematopoietic stem cells: bone marrow origin, peripheral blood source, umbilical cord blood source and placenta source. Animal sources are more extensive, such as bone marrow, umbilical cord, liver, kidney, muscle tissue or organ of young animals.

Viral diseases: diseases caused by viral infection. Common viral diseases include influenza, AIDS, measles, rubella, smallpox mumps, rubella, measles, varicella, respiratory viral infections, viral hepatitis, poliovirus, other enterovirus infections, epidemic encephalitis, epidemic hemorrhagic fever, etc.

Antiviral compounds: refer to a class of compounds that have therapeutic or prophylactic effects on viral diseases, including compounds that are being studied, developed, and commercially available. Such compounds include, but are not limited to, interferon compounds, nucleoside analogs, and the like. Such compounds may be used in combination with one or more compounds, or may be used in sequence.

### EXAMPLE 1

### Preparation of Umbilical Cord Blood Hematopoietic Stem Cells

1. Material

1.1. Equipments: ultra-clean table, horizontal centrifuge, inverted microscope and ordinary microscope. Centrifugal tube and level scale, blood collection bag.

1.2. Reagents: Meglumine diatrizoate (Sigma Co.), Hydroxymethyl cellulose (Sigma Co.), Cesium chloride (Sigma Co.), Sucrose (Ficoll-400, Phamacia Co.), Potassium dihydrogen phosphate, Sodium hydroxide (both Guangzhou Chemical Reagent Plant).

1.3. Lymphocyte separation solution (Shanghai Reagent Factory No. 2) with specific gravity of 1.076 g/ml.

1.4. Cell culture medium: DMEM medium with high sugar content of 10% calf serum (Gibco Co.).

2. Method

2.1. Cell preparation: 10 mL of cord blood plus 10 mL of stem cell separation solution per tube was added. Centrifuged at 20° C. at 1500 r/min for 15 min. The liquid in the centrifuge tube was divided into four layers, and the white cloud nucleated cells layer between the serum layer and the separation layer was absorbed. Wash with PBS, centrifuge at 800 r/min for 4 min, and count the cells.

2.2. Cell survival rate analysis: Trypan blue staining, living cell counting. Wright's staining and nucleated cells analysis.

2.3. Detection of flow cytometry: After washing the cells with PBS, PE-CD34, PE-CD133, PE-CD90, PE-CD44, PE-CD45 and monoclonal antibodies were added into the cell suspension suspended by PBS, respectively. The cells were incubated at 4° C. for 40 min, then washed and detected by the flow cytometry. At the same time, PE-IgG1 and FITC-IgG1-labeled negative control antibodies were set, and monoclonal clonal anti-stem cells were added as a blank control.

3. Results

3.1. The survival rate of cells obtained by Trypan Blue staining was above 90%.

3.2. Cell count:

Number of cells obtained per ml of bone marrow=(Number of cells per ml×the total amount of cell suspension)/(The amount of bone marrow blood was added to each centrifuge tube) Formula

Note: In this experiment, the amount of bone marrow blood added in each centrifuge tube was 10 mL.

According to the cell count results after calculation, the number of cells obtained from cord blood separation was 3.46×10⁵ per mL of umbilical cord blood.

Flow cytometric detection shows that the proportion of CD34+, CD 133+ cells in umbilical cord mononuclear cells (UC-MNCs) should be greater than 1.0%.

### EXAMPLE 2

### Preparation of Bone Marrow Hematopoietic Stem Cells

1. Material

1.1. Equipments: ultra-clean table, horizontal centrifuge, inverted microscope and ordinary microscope. Centrifugal tube and level scale, blood collection bag. Flow cytometry, drying box, glove and surgical garment.

1.2. Reagents: Meglumine diatrizoate (Sigma Co.), Hydroxymethyl cellulose (Sigma Co.), Cesium chloride (Sigma Co.), Sucrose (Ficoll-400, Phamacia Co.), Potassium dihydrogen phosphate, Sodium hydroxide (both Guangzhou Chemical Reagent Plant).

1.3. Cell culture medium: DMEM medium with high sugar content of 10% calf serum (Gibco Co.).

1.4. Lymphocyte separation solution (Shanghai Reagent Factory No. 2) with specific gravity of 1.076 g/ml.

2. Method

2.1. Add 20 ml bone marrow into phosphate buffer solution (PBS) and centrifuge it gently and evenly. Centrifuge it at 1500 r/min in a centrifuge at room temperature for 15 min. The liquid in the centrifuge tube was divided into four layers, and the intermediate cell layer rich in nucleated cells was carefully sucked, washed with PBS, and centrifuged at 800 r/min at room temperature for 4 minutes. Cell counted. Trypan blue staining, living cell count. Wright's staining and nucleated cells analysis.

2.2. Bone Marrow Stem Cells Were Identified by Rhodamine Rejection Test

The procedure for rhodamine staining was as follows: 1×10⁶ for freshly isolated cells. The cells were inoculated and cultured on the culture plate for 2 hours, and 1 μg Rhodamine 123 was added and incubated at 37° C. for 1 hour, then the extracellular Rhodamine 123 was washed off with PBS, and the intracellular distribution of Rhodamine 123 was observed by flow cytometry.

3. Results

3.1. The survival rate of cells obtained by placental blue staining was more than 90%.

### Cell Count

Number of cells obtained per ml of bone marrow=(Number of cells per ml×the total amount of cell suspension)/(The amount of bone marrow blood was added to each centrifuge tube) Formula

Note: In this experiment, the amount of bone marrow blood added in each centrifuge tube was 5 ml.

After calculation, the cell count results showed that the number of cells obtained per ml of bone marrow was 1.80×10⁶.

3.2. Wright's staining result: The isolated cells were mainly stem cells, which were small, such as the size of red blood cells, with deep nuclear staining and smaller cytoplasm.

3.3. Results of Rhodamine 123 Rejection Test

According to the characteristic that the stem cells do not actively absorb the fluorescent dye rhodamine 123, the Rhodamine 123 rejection test can be used to identify the stem cells in the bone marrow blood.

The results showed that the proportion of Rhodamine 123 cells was 3.62%, which indicated that the stem cell isolate could effectively isolate and enrich the hematopoietic stem cells from bone marrow blood.

### EXAMPLE 3

### Preparation of Bone Marrow Hematopoietic Stem Cells From Neonatal Mice

1. Materials
1.1. Reagents and solvents:
1) Sterilization water for injection, production source: self-made;
2) Normal saline and Hank's buffer;
3) Alcohol;
4) Surgical instruments.

1.2. Neonatal C57BL/6 (CD45.1) healthy mice, which were purchased from Guangdong Provincial Laboratory Animal Monitoring Center.

2. Method

Select the neonatal C57BL/6 (CD45.1) health mice, execution, alcohol disinfection, the mice bone marrow cells were collected from the bone marrow suspension under the sterile conditions, then the filtered cells suspension was centrifugated at 500 g for 5 minutes at the room temperature to remove the supernatant after collecting cells, add 2 mL RBC pyrolysis liquid suspension cell, incubated at room temperature about 10 minutes for the red blood cells to crack, the suspension cell were washed again, centrifugated, collecting the cells.

Resuspend the cells, then add the antibodies of the lineage cocktail (1:100), Sca-1 (1:100), c-kit (1:100), and incubate them in dark at 4° C. for 30 min. After centrifugation, supernatant removal, cell suspension was resuspended, and cell suspension was filtered by a 40 μm membrane. The hematopoietic stem cells of LSK (Lin-Sca-1+C-KIT+) were analyzed by flow cytometry.

3. Results

3.1. Hematopoietic stem cells of 10 neonatal mice were isolated and the cell number was 2.1×10⁷ by cell count, and then the cells were frozen for storage.

3.2. Flow detection results: the percentage of CD34+ cells was 3.99% and the percentage of CD133+ cells was 4.64%.

### EXAMPLE 4

### Preparation of the Hepatic Hematopoietic Stem Cells in the Fetal Rats

### Experimental Purpose: To Isolate Hematopoietic Stem Cells From the Rat Embryonic Liver Tissue

1. Materials

1.1. Reagents and Solvents

1) Sterilization of water for injection, production source: self-made;

2) Normal saline and Hank's buffer;

3) Alcohol;

4) Surgical instruments.

1.2. Animals and the Feeding Conditions

Animals: 7 SD rats in SPF class; 6-8 weeks; 130˜200 g, female, pregnant, from Guangdong Laboratory Animal Monitoring Center;

1.3. Instruments

1.3.1 Electronic Scale: Mettler Toledo AB104S;

1.3.2 Freezing slicer: Leica, CM1900;

1.3.3 Bio-fluorescence microscope: Leica, DM5000B;

1.3.4 Automatic biochemical analyzer: Beckman Counter CX5;

1.3.5 Desktop high speed low-temperature Centrifuge: Ependoff 5804R;

1.3.6 Cell Counter: Invitrogen, Countess.;

1.3.7 Microplate Reader: Thermas, Multiscan FC.

[symbol]

[symbol] [symbol] [symbol] , [symbol] , [symbol] [symbol] , [symbol] ; [symbol] 1: 2 [symbol] DMEM [symbol] , [symbol] ,

[symbol]

[symbol] [symbol] , [symbol] 0.01% [symbol] 1 [symbol] , [symbol] 0.1% [symbol] 20˜40 min [symbol] , 40 [symbol] [symbol] ,

[symbol]

[symbol] HSCs [symbol] ( [symbol] 90% [symbol] DMEM/F12, 10% [symbol] DMSO) [symbol] [symbol] , [symbol] , [symbol] [symbol] 4 [symbol] 24 [symbol] ,

[symbol]

[symbol]

[symbol]

2. Method

2.1. SD rats at about two weeks of pregnancy were killed and the liver of the fetal rats was taken, and the liver tissue was separated and weighed under sterile conditions; DMEM medium was added 1:2, tissues were cut into pieces and centrifuged.

2.2. Fetal liver tissue was digested with 0.01% collagenase for 1 hour, and then digested with 0.1% trypsin for 20˜40 min, and then stop digestion. Single cell suspension was made by filtration with 40-mesh sieve, and the live cells were counted.

2.3. HSCs were suspended with a frozen solution (DMEM/F12 with volume fraction of 90%, DMSO with volume fraction of 10%), cooled at a constant rate, and finally frozen in liquid nitrogen. Resuscitation was performed at 4 weeks and 24 weeks, respectively. Survival cell proportion was counted and flow cytometry was performed.

3. Results

3.1. The wet weight of liver tissue was 1.95 g/10 fetal rats.

3.2. Isolated cell count, the total number of cells was 0.4×10⁶.

3.3. The survival rate of cryopreserved cells after resuscitation was 93%, and the proportion of CD133 cells and CD34 cells was 2.3% and 3.6% by the flow cytometry.

### EXAMPLE 5

### Preparation of Pig Bone Marrow Hematopoietic Stem Cells

1. Material

1.1 Equipment: Ultra-clean table, Horizontal centrifuge, Inverted microscope and ordinary microscope. Centrifugal tube and balance scale, bone marrow puncture needle and blood collection bag. Flow cytometer, drying oven, gloves and operating gowns.

1.2. Reagents: Meglumine diatrizoate (Sigma Co.), Hydroxymethyl Cellulose (Sigma Co.), Cesium Chloride (Sigma Co.), Sucrose (Ficoll-400, Phamacia Co, Ltd.), Potassium Dihydrogen Phosphate, Sodium Hydroxide (Guangzhou Chemical Reagent Factory), Rhodamine 123 (Sigma Co.).

1.3 BD ProCount Progenitor Cell Enumeration Kit (BD, Batch No. 340498).

1.4. Lymphocyte separation solution (Shanghai Reagent Factory No. 2), specific gravity is 1.076 g/ml.

1.5. Cell culture medium: DMEM medium with high glucose containing 10% calf serum (Gibco).

1.6. Pigs used in the experiment were all newborn Potentilla anserina pigs, male, 0.5 kg weight, provided by Fangcun Slaughter house, Guangzhou.

2. Method

2.1. Bone marrow extracted from the experimental pigs were performed as the following steps:

The experimental pigs were anesthetized i.p. with 30 mg/kg sodium pentobarbital. high pressure sterilization All surgical instruments were sterilized with a high pressure disinfection. The operation room were disinfected with the ultraviolet disinfection, the pig legs fur were electrically cut off, the body surface of experimental pigs were disinfected with 75% alcohol. After the local anesthetized with the lidocaine, the 30 ml bone marrow of the pigs were extracted in iliac.

2.2. After successful bone marrow extraction, the pig bone marrow cells were isolated by the following steps:

The bone marrow was added into the phosphate buffer solution (PBS), mixed and centrifuged at 1500 r/min for 15 minutes in a normal temperature centrifuge. The supernatant was discarded and the precipitate was gently resuspended in the phosphate buffer solution (PBS) to obtain the cell suspension. The cell suspension was slowly added unto the surface of the stem cell isolating solution in a 2:1 ratio, and centrifuged at 2500 r/min at 20° C. for 10 min. The liquid in the centrifuge tube was divided into four layers, and the intermediate cell layer rich in nucleated cells was carefully aspirated, washed with PBS, and centrifuged at 800 r/min at room temperature for 4 minutes. Living cells were counted with Trypan Blue staining. the nucleated cells were analyzed with Wright's staining.

2.3. The Rhodamine rejection test was used to identify the porcine bone marrow stem cells.

The procedure for rhodamine staining was as follows: 1×10⁶ cells were freshly isolated and incubated on the culture plate for 2 hours. 10 μL of 1 μg/mL Rhodamine 123 was added. After incubation at 37° C. for 1 hour, the extracellular Rhodamine 123 was washed off with PBS, and the intracellular distribution of Rhodamine 123 was observed by flow cytometry.

2.4. Porcine bone marrow stem cells were identified by the stem cell counting kit and the flow cytometry

The freshly isolated cells were added to the Eppendoff tube (10⁶ cells/tube), centrifuged at 1500 rpm for 5 min, washed once with PBS, added with 1 ml cold acetone, and fixed at 0-4° C. for 8 min; Centrifugated, abandon supernatant, the first antibodies (CD34, CD45) were added, blending at 37° C., reacted for 45 minutes, 1500 RPM centrifugated for 10 minutes, PBS washed for three times. Centrifugated, the supernatant were abandoned, added the second antibody marked IgG, blending 37° C. allowed to react for 30 minutes, PBS washing twice, depending on the amount of sediment were added with the PBS as the amount of sediment after centrifugal, the cell suspensions were made, the flow cytometry instrument were tested.

3. Results

3.1. According to Trypan Blue staining, the survival rate of the cells was larger than 90%, the cells counted as above.

3.2. Results of Rhodamine 123 rejection test.

Considering that the characters of the stem cells do not actively absorb the fluorescent dye rhodamine 123, the Rhodamine 123 rejection test can be used to identify the stem cells in the bone marrow blood.

3.3. The results of the porcine bone marrow stem cells were identified by the stem cell marker kit and flow cytometry

The stem cell marker kit is mainly used to identify the bone marrow hematopoietic stem cells, in which CD34 is used to mark hematopoietic stem cells, CD45 is used to mark lymphocytes, PI is mainly used to mark nucleated cells.

The percentages of CD34, CD45 and nucleated cells in cell suspensions obtained from the stem cell isolating solutions were determined by flow cytometry, as shown in Table 1.

TABLE 1
Proportion of cells with various markers isolated from the porcine
bone marrow cells by the stem cell isolating solution

| | Specific gravity of | Proportionof | Proportion of | Proportion of |
|---|---|---|---|---|
| Tube | separation solution | Nucleated Cells | CD34+ cells | CD45+ cells |
| No. | (g/ml) | (%) | (%) | (%) |
| 1 | 1.076 | 52.31 | 0.15 | 45.8 |
| 2 | 1.080 | 45.35 | 1.57 | 41.57 |
| 3 | 1.086 | 44.58 | 2.37 | 40.96 |

Tube No. 1 is the lymphocyte isolation fluid, while as Tube No. 2-3 were the stem cell isolating solutions.

The results showed that the number of CD34 positive cells obtained from the stem cell isolating solutions with the specific gravity of 1.086 was 12 times more than that from the lymphocyte isolation fluid.

3.4. The cells were cultured for 1-7 days and observed under a phase contrast microscope. The results were as follows: the newly isolated bone marrow mononuclear cells were spherical and dispersed in the culture medium, and there were a small number of hematopoietic cells. The morphology of the remaining cells discarding the superannuates after change the medium, was similar to that of bone marrow stromal cells. The cells grew adherent to the cell wall and the shapes were fusiform.

After centrifugation, the non-adherent cells were changed into a new culture medium and cultured for the later 7 days, the clumped cells could be seen. The cells obtained from the stem cell isolating solutions were clumped and grew actively.

4. Conclusion

Compared with the peripheral blood, less cells were isolated and purified from bone marrow blood, and the separation process was relatively complex, the cells were uniform in size and had a good refraction under the microscope.

Porcine bone marrow hematopoietic stem cells can be successfully isolated by the stem cell isolating solution. Bone marrow blood is 2.7×10⁸, the results of flow cytometry showed that the hematopoietic stem cells were CD34+ cells (2.37%) and CD133+ cells (1.73%), the proportion of hematopoietic stem cells could reach more than 1%.

### EXAMPLE 6

### Preparation of the Liver Hematopoietic Stem Cells of Small Pigs

1. Experimental purpose: To compare the separation of hematopoietic stem cells from neonatal pig liver by different separation methods, enzymes and separation liquids.

2. Experimental equipment: 2 mL pipette, 50 mL centrifuge tube, DMEM/F12, PBS, II type collagenase, trypsin, DNA enzyme, protease, polyvinylpyrrolidone, separation liquid.

3. Experimental Procedures

3.1 Separation

Liver tissue was isolated from the neonatal sucking pigs under aseptic conditions, then added into DMEM medium at a rate of 1:2, the tissue was cut into pieces and centrifuged.

3.1.1 Manual method: The tissues were separated and weighed. Cut into pieces about 0.1 mm³ with scissors, add PBS at 1:10 (trypsin or collagenase can be added at the same time), centrifuge at 250 g for 5 minutes, removed the supernatant, and the precipitation were washed with 0.1 M/L PBS for three times; collect the tissues for further use.

3.1.2 Electro-mechanical method: The tissues were separated and weighed. Add PBS at 1:10 (trypsin or collagenase can be added at the same time) in a tissue grater or homogenizer, and homogenate for 1 min at 7000 RPM. The homogenate was centrifuged at 250 g for 5 minutes, the supernatant was removed, and t the precipitation were washed with 0.1M/L PBS for three times; collect the tissues for further use.

3.2 Digestion

3.2.1 Collagenase and Trypsin Digestion

3.2.1.1 Centrifugated 250 g for 5 min, the precipitation was digested with 0.01% collagenase of 10 times volume at 37° C. for 30 min. Cells were collected, counted and the survival rate of the cells were detected.

3.2.1.2 Wash, then digest with 0.01% trypsin of 10 times volume for 30 minutes, and observe the digestion effect; Washed with PBS for 3 times, cells were collected, filtered through a 40-mesh sieve to make single-cell suspension, counted and survival rate detected.

3.2.2 Multi-Enzyme Digestion

The precipitation was washed out with 10 times volume of 0.1 M/L PBS solution, and then digested at 37° C. for 30 minutes with 0.01% collagenase, 0.02% proteinase K and 0.005% DNA enzyme. The digestion effects were observed according to the digesting time.

3.3 Purification

Add the stem cell isolating solution into 50 ml centrifuge tubes, and take the above cells at 1×10⁷/ml of the cell suspension about 10 ml, carefully added to the solution (The isolating solution: the cell suspension volume was 1:1), centrifuged, took the cells at the interface between the isolating solution and the supernatant, washed them with 0.1M/L PBS solutions for 3 times, counted the cells and frozen.

3.4 Resuscitation of the Frozen Cells

The frozen cells were suspended in cryopreservation solution (90% DMEM/F12, 10% DMSO), cooled at a constant rate, and finally frozen in liquid nitrogen. Resuscitation was performed at 4 and 24 weeks, and the proportion of living cells was counted. 4. Results: The weight of the liver was 13.58 g, and the total number of cells isolated was 1.9×10⁷, in which, the hematopoietic stem cells were CD34+ cells (2.69%) and CD133+ cells (1.43%).

### EXAMPLE 7

### Preparation of the Bone Marrow Hematopoietic Stem Cells From Newborn Chicks

Experimental purpose: To isolate the hematopoietic stem cells from the bone marrow of newborn chicks.

1. Materials
1.1. Reagents and solvents:
1.1.1. Sterilization water for injection, the production unit: self-made.
1.1.2. Normal saline and Hank's buffer;
1.1.3. Alcohol;
1.1.4. Surgical instruments.
1.2. Newborn healthy chickens purchased from Baiyun Chicken Farm, Guangdong Province.

2. Methods

Three newborn healthy chicks were selected, killed, disinfected with alcohol, bone marrow cell suspensions were taken out under the aseptic conditions, the filtered cell suspensions were centrifuged at 500 g at room temperature for 5 min, the supernatants were removed, the cells were collected and resuspended, the cell suspensions were filtered through a 40 μm filter membrane, and the survival rates of HSCs were counted.

3. Results

The total number of mononuclear cells in the bone marrow blood of three chicks was 1.2×10⁸, CD34 and CD133 cells were not detected by flow cytometry, which may be related to the different surface marker components of the chicken HSCs.

### EXAMPLE 8

### Experimental Study of Umbilical Cord Blood Hematopoietic Stem Cells Inhibiting HBV Replication in Ducks

Rationale: Duck hepatitis B is a model of hepatitis that naturally infects ducks and can lead to chronic hepatitis and cirrhosis. The antiviral activity of the drugs can be determined by detecting the contents of HBsAg, HBeAg and HBV DNA in duck serum. In this example, the hematopoietic stem cells from the umbilical cord blood were transplanted into ducks in vivo to observe their inhibitory effect on hepatitis B virus.

1. Materials and Methods

1.1 Reagents: 32P-dCTP and notched translation kit (Promega); Fish sperm DNA, bovine serum albumin (Sigma products).

1.2 Animals: 6 female ducks, purchased from the market; One-day-old Beijing duck, purchased from Likang Agriculture and Industry United Company, Guangzhou, Guangdong Province.

1.3 Screening of positive sera: the 6 ducks were sampled under aseptic conditions to separate the sera. Conventional PCR results showed that 3 ducks were positive for PCR amplification, with 3 positive bands, while no positive bands were found in negative control. The serum of duck with higher virus titer was selected as the positive serum for future usage.

1.4 Preparation of the duck hepatitis B model: the positive duck serum was taken to infect 1-day-old ducks. One hundred healthy ducklings were collected on hatching day, 95 ducklings were injected with 100 μL positive duck serum via leg vein, 5 ducklings were not treated as the normal control group. Detection of hepatitis B infection rate in ducks: venous blood samples were collected at the second week after infection, and DHBV was detected by PCR. Results Of the 95 ducks infected with DHBV positive serum, 60 were positive, and all the 5 normal control ducks were negative. The duck hepatitis B model was successfully constructed. Thirty ducks with viral serum titer greater than 10000 were selected for the further experiment.

1.5 Thirty positive ducks were divided into three groups, as shown in Table 2:

TABLE 2
Experimental grouping

| Lamivudine Group | − | + | − |
|---|---|---|---|
| Stem cells Group | − | − | + |
| Number of animals | 10 | 10 | 10 |

Note:
“+” means adding the corresponding drug, and “−” means not adding the corresponding drug.

The treatment was started 2 weeks after infection, and the titer of duck hepatitis B virus was detected by semi-quantitative PCR after 8 weeks of continuous treatment.

1.6 PCR reaction: 50 μL duck serum was added with 50 μL lysate, boiled at 100° C. for 10 min, centrifuged rapidly, and then placed on ice as a template. In the conventional PCR reaction, the positive serum was used as the positive control, and a total of 5 positive controls were set for each reaction. The blank control contained all the components required for RT-PCR, but no template was added.

1.7 Gel electrophoresis analysis: PCR reaction products were separated by the agarose gel, and the results were quantitatively analyzed on the multifunctional imaging analysis system.

1.8 Pathological examination: routine paraffin section and HE staining were performed to observe the histomorphology of the degrees of inflammation and degrees of degeneration of the liver.

1.9 Statistical analysis: All the results were statistically analyzed by SPSS software package, and P<0.05 was considered to be significant.

2. Results

2.1 After the lamivudine and the stem cell treatment, hepatocyte degeneration and inflammation were significantly reduced in both the stem cell and lamivudine groups compared with the control group.

2.2 Changes of virus titers in ducks before and after lamivudine and stem cell treatment were shown in Table 3.

TABLE 3
Effects of the lamivudine and the stem cell therapy on
hepatitis B virus titer in ducks (n = 10, χ ± SD)

| Lamivudine | − | + | − |
|---|---|---|---|
| group | | | |
| Stem cells | − | − | + |
| group | | | |
| DHBV drop | 12867.6 +/− | 3896.4.2 +/− | 8879.6 * |
| degree | 4023.1 | 1108.7 * * | 2367.9 mm |

Note:
“+” means adding the corresponding drug, and “−” means not adding the corresponding drug.
* means p < 0.05;
** indicates P < 0.05 (compared with normal saline group).

The results showed that after the treatment, the low titer of DHBV was found in the lamivudine treatment group. After the stem cell treatment, the titer of DHBV in duck serum decreased to a certain extent, but the antiviral effect was not as good as that in the lamivudine treatment group.

3. Conclusion

3.1 Lamivudine and stem cell therapy can significantly reduce the pathological changes of hepatitis B in ducks.

3.2 Cord blood stem cell therapy has a certain inhibitory effect on duck hepatitis B virus, but the effect is not as obvious as lamivudine.

### EXAMPLE 9

### Therapeutic Effect of Umbilical Cord Blood Hematopoietic Stem Cells on Hepatitis Virus Infection in Mice

Mouse hepatitis virus (MHV) is a common pathogen of mouse infections. MHV is a member of the genus Coronavirus of the Coronavirus family, and its genome is single-stranded RNA. MHV is often used as a model for coronavirus research.

Cord blood stem cells (CBC) have been a hot research topic in recent years and have a great value in clinical application.

1. Materials and Methods

1.1. Animal Grouping and Stem Cell Intervention

SPF NIH female mice were used to conduct three experiments in parallel. The animals were randomly divided into three groups of 20 animals per group. One group acted as a control group and was infected only with MHV, One group, as treatment group 1, was injected with stem cell suspension once a day for 3 consecutive days, 3 days before the stem cell intervention, and then was infected with MHV. One group was treated as group 2, after MHV infection and then injected with stem cell suspension. The first injection was given 24 hours after infection, at intervals of 24 hours, for a total of three doses. They were injected into the tail vein with 50 microliters of stem cell suspension. Mice infected with MHV were inoculated intraperitoneally with a dose of 0.2 mL, and the virus content was about 10⁶ TCID50.

Note: The primary purpose of this preliminary experiment was to evaluate the improvement of the mortality of mice infected with MHV by stem cells, so the dose and times of the stem cells were used as much as possible. The cell suspension is set to a high concentration of 10⁸/ml is even higher. The number of injections was set to 3 times.

1.2. Observation Indicators

Previous studies have shown that NIH mice infected with MHV have a high mortality rate on day 4-6, and no mice die after 7 days. The clinical manifestations and mortality of the three groups of mice were observed. The mortality of mice infected with MHV was improved by the stem cells, and the key point was the first 7 days after infection.

2. Results

On the 8th day after the virus infection, 9 mice died in the control group and 6 mice died in the stem cell group after the virus infected. There were no deaths in the stem cell group. No deaths were observed at 9, 10, 11 and 12 days after infection.

3. The Conclusion

In this study, the animals died on the 8th day after virus infection. The stem cell injection after virus infection can protect the animals, and the extraction of injected the stem cells can protect the animals against virus infection. Subsequent observation showed no deaths. This study was able to observe that the stem cells have a good protective effect against the virus infection in animals, but further experiments need to be repeated to confirm this effect.

### EXAMPLE 10

### Protective Effect of Umbilical Cord Blood Hematopoietic Stem Cells on Mice Infected With Influenza Virus

1. Experimental purpose: To test the protective effect of stem cells on mice infected with influenza virus.

2. Material

Virus: A/PR8/34 (H1N1).

Umbilical cord blood stem cells and control cells (mesenchymal stem cells): Provided by Guangzhou Cedicine Biotechnology Co., Ltd.

Cells: Madin-Darby Canine Kidney Cells (MDCK).

3. Method

Ninety SPF BALB/C male mice aged 6 weeks were randomly divided into five groups: the normal control group, the virus group, the high-dose (the cord blood stem cells, 1.0×10⁸/kg) and the low-dose groups (the cord blood stem cells, 1.0×10⁷/kg) were injected into 0.2 ml normal saline through caudal vein, control group (mesenchymal stem cells at 1.0×10⁸/kg was injected in 0.2 ml normal saline through caudal vein). Except the normal control group, the other groups were injected with the same amount of culture medium. Treatment was performed on day 3 of infection, with either stem cells or control cells injected into a caudal vein (one dose at one time point).

3.1 Death Protection

The mice were observed continuously for 15 days, the body weight and death number of the experimental animals were recorded every day, and the survival rate and average survival time were calculated according to the results.

3.2 Detection of Pulmonary Pathology

Lung tissue was collected from half of the mice on the 5th day after infection, and lung index was calculated and pathological sections were made to evaluate the status of infection model pneumonia. After lung tissue was removed without bronchoalveolar lavage, blood stains on the surface were rinsed with normal saline and fixed with 4% paraformaldehyde for 24 h, followed by rinsing paraformaldehyde with PBS and then fixation with 4% sucrose solution for 24 h. Paraffin immersion and embedding, sections were stained with HE (this process was automatically completed by the machine).The degree of lung tissue injury, inflammatory cell infiltration and other conditions were observed under optical microscope after modeling, and the images were saved with digital photography integrated system.

3.3 Detection of the Virus Titer in the Lung Tissue

Remove the lung tissue and put it into the EP tube sterilized by high temperature and autoclaved, add 1 mL frozen PBS liquid, homogenize the lung tissue with electric homogenizer (on ice), centrifuge at 3000 RPM for 5 minutes, and take the supernatant. The MEM medium containing 2 times penicillin, streptomycin, amphotericin and 1.5 g/mL TPCK-treated trypsin was added with gradient 10 times dilution, 100 μL/well diluent of MDCK cells prepared in 96-well plate was added. The cytopathic effects were observed 48 hours later, and TCID50 was calculated by Reed-Muench method.

4. Results

4.1 Effects of the umbilical cord blood stem cells on the lung weight of mice infected with influenza virus: the results are shown in Table 4.

TABLE 4
Impact on lung index in mice infected with influenza virus

| | | | Lung index, g/body | Inhibition |
|---|---|---|---|---|
| Group | Dose | Number | weight 10 g | rate (%) |
| Normal | — | 10 | 0.80 +/− 0.09 | — |
| control group | | | | |
| Virus group | — | 20 | 1.31 +/− 0.26^{#} | — |
| High dose | 1.0 × 10⁷/kg | 20 | 0.91 +/− 0.05* * * | 30.5 |
| group | | | | |
| Low dose | 1.0 × 10⁸/kg | 20 | 0.98 +/− 0.06* * | 25.2 |
| group | | | | |
| Control group | 1.0 × 10⁸/kg | 20 | 1.09 +/− 0.08*^{, ∘} | 16.8 |

Note:
^{#}P < 0.001, indicating successful modeling; Compared with model group, *P < 0.05, * *P < 0.01, * * *P < 0.001; P < 0.01 compared with the high dose group.

The results in Table 4 indicated that the lung index of mice infected with influenza virus could be significantly decreased by the medium and high doses of the umbilical cord blood stem cells (P<0.01˜0.001), and there was also a significant difference compared with mesenchymal stem cells (P<0.01).

4.2 Protection against death of mice infected with influenza virus: The results are shown in Table 5.

TABLE 5
Death protection of mice infected with influenza virus

| | | Death | Mortality rate | Mean days of |
|---|---|---|---|---|
| Group | Number | number | (%) | survival |
| Normal control | 10 | — | — | >14 |
| group | | | | |
| Virus group | 20 | 17 | 85^{#} | 6.8 |
| High dose group | 20 | 3 | 15* * * | 9.42 |
| Low dose group | 20 | 11 | 55* * | 8.26 |
| Control group | 20 | 10 | 50* | 9.08 |

Note:
Compared with model group, *P < 0.05, * *P < 0.001.

The results in Table 5 suggested that the high dose of umbilical cord blood stem cells group could significantly reduce the mortality of mice infected with influenza virus (P<0.05), and prolong the survival days of infected mice, and the effect was better than that of the low dose group and the mesenchymal stem cells group, with a clear dose-effect relationship.

5. Conclusion:

Umbilical cord blood stem cells can significantly reduce the mortality of mice infected with influenza virus (P<0.05), prolong the survival days of infected mice, and also significantly reduce the pulmonary inflammation and lung index caused by influenza virus.

### EXAMPLE 11

### The therapeutic Effect of the Porcine Hematopoietic Stem Cells on Porcine Influenza and Severe Pneumonia

1. Experimental purpose: To observe the effect of porcine hematopoietic stem cells on porcine influenza and severe pneumonia.

Twenty-four boars with influenza complicated with severe pneumonia were selected and treated with the hematopoietic stem cells to observe the therapeutic effect.

2. Materials and Methods

2.1 Preparation of the porcine hematopoietic stem cells, see example 5 of this patent, prepared by Guangzhou Cedicine Biotechnology Co., Ltd.

2.2 Diagnosis and Group

Swine flu has been reported in a pig farm in Foshan. The sick pigs showed symptoms such as not eating, depressed, no walking, their body temperature rising and breathing faster. More than 120 boars nose liquid thick silk, with rust color, breathing sound thickened, occasionally can hear cough, chest auscultation rales. According to epidemiological survey, clinical symptom, the pigs in 100-150 kg diagnosed with swine flu and the onset of severe pneumonia caused by mixed infection of 36 boars. In the treatment group, add the hematopoietic stem cell treatment in the ordinary course of traditional Chinese medicine (TCM) therapy: high dose group of 12, intravenous injection, the dose of 1.0×10⁷/kg body weight; in low-dose group were injected intravenously at a dose of 1.0×10⁶/kg body weight. Control group, ordinary Chinese medicine treatment, Shuanghuanglian injection, Henan Fusen Pharmaceutical Co., Ltd.

2.3. Methods: Isolation and treatment were carried out on the sick pigs. 36 boars were bound with the turbinate bone of the pigs with a large nylon rope. Stem cell high-dose group, 12 boars were treated with hematopoietic stem cells (1.0×10)⁷/kg body weight was added into normal saline 100 ml for intravenous injection+Shuanghuanglian injection 50 ml for intravenous injection, once. Stem cell low-dose group (12 boars) was treated with hematopoietic stem cells (1.0×10)⁶/kg body weight was added into normal saline 100 ml for intravenous injection+Shuanghuanglian injection 50 ml for intravenous injection, once. In the control group, only Shuanghuanglian injection 50 ml was injected intravenously, once.

2.4. Curative effect criteria: normal body temperature, normal appetite, disappearance of respiratory symptoms and no outflow of thick nasal fluid are considered as cured.

3. Results: The therapeutic effect of the porcine hematopoietic stem cells on porcine influenza and severe pneumonia was observed in Table 6.

TABLE 6
Curative effect of porcine hematopoietic stem
cells on the treatment of porcine influenza and
severe pneumonia (number of cures)

| | | Ssecond | Third | Fourth | Eight | Number | Cure |
|---|---|---|---|---|---|---|---|
| | Number | day | day | day | day | of cure | rate % |
| High | 12 | 4 | 6 | 2 | | 12 | 100** |
| dose group | | | | | | | |
| Low | 12 | | 6 | 4 | 1 | 11 | 92* |
| dose group | | | | | | | |
| Control | 12 | | 2 | 4 | 3 | 9 | 78 |
| group | | | | | | | |

Note:
Compared with the control group, *P < 0.05,
**P < 0.001.

It can be seen from Table 6, the stem cells can promote the cure of swine influenza and severe pneumonia, with a significant difference of 100% and 92% in the low-dose group, compared with 75% in the control group, and can accelerate the recovery of swine influenza.

4. Conclusion:

Stem cells can promote the cure of swine influenza and severe pneumonia, and accelerate the recovery of swine influenza.

### EXAMPLE 12

### Effect of Umbilical Cord Blood Stem Cells on the Treatment of Influenza and Severe Pneumonia in the Elderly People

Influenza (abbreviation flu), it is an acute fever respiratory tract infectious disease that causes by flu virus, spread via fluttering foam. The typical clinical expression is the whole-body toxic symptoms such as the protuberant fear cold, high heat, headache, whole body ache, and weakness. The most striking feature of epidemiology is the sudden outbreak, rapid spread and wide coverage. Influenza often gets worse 2 to 4 days after the onset of illness or in the convalescence period, with high fever, severe cough, purulent sputum, dyspnea and rale of wet lungs. Infants, the elderly, patients with heart and lung diseases and other chronic diseases or immunocompromised patients can be complicated with pneumonia, and the prognosis is poor.

1. Materials and Methods

1.1 General Information

A total of 140 inpatients who met the clinical diagnostic criteria for severe influenza were collected. Among them, 74 were males and 66 were females. Their age ranges from 60 to 83, with an average age of 69. 121 patients (86.43%) had high risk factors for influenza, including 62 patients with chronic diseases such as respiratory system and cardiovascular system, 12 cases were obese. By random number table method, 70 cases were divided into the stem cell group (treatment group) and combined treatment group (control group).

There were 35 males and 35 females in the treatment group. Age ranged from 66 to 83 years old, median age 71 years old; There were 39 males and 31 females in the control group. The age ranges from 60 to 82 years, the median age is 70 years, and the gender and age groups are balanced.

1.2 Diagnostic Criteria

1.2.1 Diagnosis of the Severe Influenza:

The criteria for the diagnosis of influenza as set out in the Guidelines for the Diagnosis and Treatment of Influenza (2018 Edition) and meets one of the following critical criteria:

(1) Rapid dyspnea, cyanosis of the mouth;

(2) Mental changes such as retarded reaction, lethargy, restlessness and convulsion;

(3) Severe vomiting, diarrhea and dehydration;

(4) Imaging examination showed signs of pneumonia;

(5) The levels of myocardial enzymes such as creatine kinase (CK) and creatine kinase isoenzyme (CK-MB) increased rapidly;

(6) The original basic diseases were significantly aggravated.

1.2.2 Diagnosis of the Bacterial Pneumonia Secondary to Influenza

Severe influenza patients with persistent high fever >for 3 days, accompanied by severe cough, purulent sputum, dyspnea, lung wet rales and pulmonary consolidation signs; Secondary bacterial pneumonia can be considered if any of the following detection indicators exceed the upper limit of normal value:

(1) total number of peripheral white blood cells;

(2) Peripheral blood neutrophils;

(3) C-reactive protein.

1.3 Inclusion Criteria

Meet the diagnostic criteria of influenza bacterial pneumonia, age ≥60 years old; Consent to participate in the stem cell clinical study, and signed the informed consent.

1.4 Exclusion Criteria

People with mental disorders.

Participated in other influenza drug clinical researchers for nearly 1 month.

Meet one of the critical disease diagnostic criteria: respiratory failure;

Toxic shock of infection;

Multiple organ dysfunction;

There are other serious clinical conditions that require monitoring.

Pregnant women.

1.6 Treatment Regimens

1.6.1 Control Group

With reference to the influenza a (H1N1) diagnosis and treatment schemes (2010 edition), and the diagnosis and treatment of influenza guide (2018 edition) “to carry on the comprehensive treatment, including antiviral (oseltamivir, for example) and symptomatic support, according to different patients to choose antibiotics (penicillin, for instance, archie, doxycycline, levofloxacin, amoxicillin, cefaclor), etc.

1.6.2 Treatment group: the umbilical cord blood stem cells (2×10⁶/kg body weight) were added on the basis of comprehensive treatment, added in 100 ml normal saline intravenously.

1.7. Observation Indexes

Lung X-ray, blood routine and C-reactive protein tests were performed to determine secondary bacterial pneumonia. Record the use of anti-influenza drugs.

1.8. Statistical Methods

Set up the central database input system, the data were input by the two persons respectively, the accuracy of the data were checked carefully. SAS9.1.3 statistical software was used for data analysis. Counting data were described in terms of frequency and percentage, and rank sum test was used. P≤0.05 was considered statistically significant.

2. Results

2.1 Comparison of the Use of Anti-Influenza Virus Drugs Between the Two Groups

Among the 70 patients in the treatment group, 46 patients (65.71%) were treated with antiviral drugs. In the control group, 54 cases were used, accounting for 77.14%.There was no significant difference between the two groups. It indicated that the antiviral treatment level of the 2 groups was the same.

2.2 Comparison of Secondary Bacterial Pneumonia Between the Two Groups

Of the 140 cases of severe influenza, 48 cases (34.28%) had secondary bacterial pneumonia. Of the 70 patients in the treatment group, 9 cases (12.86%) developed bacterial pneumonia, and of the 70 patients in the control group, 26 cases (37.14%) developed bacterial pneumonia. The incidence of bacterial pneumonia between the two groups was statistically significant (P<0.01).

3. Conclusion

Stem cell therapy can promote the recovery from severe influenza and reduce the incidence of concurrent pneumonia.
[1] Influenza diagnosis and treatment plan (2018) [J]. Chinese Journal of Infection Control, 2018, 02:181-184 (Chinese);
[2] Diagnosis and treatment plan of influenza A (H1N1) (2010) [J]. International Journal of Respiration, 2011, 31(2): 81-84 (Chinese).

### EXAMPLE 13

### Therapeutic Effect of Umbilical Cord Blood Stem Cells on Viral Encephalitis in Children

Viral encephalitis is a kind of intracranial infection of pediatrics common disease, parenchymal inflammation induced by the virus infection, the disease develops urgent, progress is fast, usually is caused by arbovirus, intestinal virus, rubella, and pure scar rash caused by virus, with life threaten when seriousness, or residue sequelae (Chen Yong, Wu Huaping, Diagnosis and treatment of viral encephalitis in children [J]. Journal of Applied Clinical Pediatrics, 2012, 27 (24):385-402 (Chinese).

In this study, we combined the umbilical cord blood stem cells with Ganciclovir in the treatment of pediatric viral encephalitis, and the results were as follows.

1. Materials and Methods

1.1 General Information

A total of 32 cases of children with the viral encephalitis admitted to Guangdong 999 Brain Hospital from January 2013 to January 2015 were collected, including 30 males and 2 females, aged from 3 months to 11 years old, with an average (7.3 ±0.6 years old).The clinical manifestations were fever in 5 cases, headache in 19 cases, vomiting in 8 cases, convulsion in 17 cases, disturbance of consciousness in 6 cases and meningeal irritation in 13 cases.

32 children were randomly divided into control group and stem cell group, 16 cases in each group. There was no significant difference in age, gender and disease between the two groups, but there was comparability.

1.2 Methods

The children in both groups were given a conventional treatment, including anti-infection, fluid replacement to maintain water and electrolyte balance, and symptomatic treatment for antishock, antipyretic and intracranial pressure reduction. In the presence of high fever, convulsions or disturbance of consciousness, dexamethasone and ganciclovir were taken orally, and the umbilical cord blood stem cells (2×10⁶/kg body weight, which added in 100 ml normal saline intravenously) were added to the experimental group.

1.3 Criteria for Efficacy Evaluation: According to the quality control standards for common diseases issued by the Ministry of Health of China, the criteria for efficacy evaluation of children were further formulated.

Significant effect: the children's symptoms and signs disappear, can resume normal work or life.

Effective: The child's symptoms and signs are reduced, but there are still some signs and residual symptoms.

NULL: No significant improvement in symptoms and signs.

The effective rate of treatment for children was statistically significant and effective.

1.4 Statistical Methods

The detected data were analyzed with the professional statistical software package SPSS26.0.

2. Results

The treatment effect of the two groups was shown in Table 7.

TABLE 7
Comparison of the time for improvement of symptoms and
signs between the two groups

| | Number | Antipyretic time | Convulsion | Regain |
|---|---|---|---|---|
| Group | of cases | (days) | control (day) | consciousness (day) |
| Control | 16 | 3.6 +/− 0.75 | 7.2 +/− 2.3 | 14.6 +/− 2.8 |
| group | | | | |
| Stem cells | 16 | 2.3 +/− 0.6 | 3.2 +/− 1.2 | 6.5 +/− 2.1 |
| group | | | | |
| P value | — | <0.05 | <0.01 | <0.01 |

In the control group, 4 cases were significantly effective, 3 cases were effective, and 9 cases were ineffective, with an effective rate of 43.75%. In the stem cell group, 10 cases were significantly effective, 5 cases were effective, and 1 case was ineffective, with an effective rate of 93.75%. The effective rate in the stem cell group was significantly better than that in the control group, with statistical significance (P<0.01).

3. Conclusion:

Cord blood stem cells combined with Ganciclovir is effective in the treatment of infantile viral encephalitis.

1. A use of hematopoietic stem cells in the preparation of a formulation for the treatment of viral diseases, wherein said viral disease is selected from one of the group: viral hepatitis, influenza, viral interstitial pneumonia, viral encephalitis and avian influenza.
use of hematopoietic stem cells in the preparation of a formulation for treating viral diseases.

2. The use according to claim 1, wherein said hematopoietic stem cells are umbilical cord blood mononuclear cells extracted from animal umbilical cord blood, and the proportion of hematopoietic stem cells should be greater than 1%.

3. The use according to claim 1, wherein said hematopoietic stem cells are bone marrow blood mononuclear cells extracted from animal bone marrow blood, and the proportion of hematopoietic stem cells should be greater than 1%.

4. The use according to claim 1, wherein said hematopoietic stem cells are single core cells in tissue blood extracted from young animal tissues, and the proportion of hematopoietic stem cells should be greater than 1%.

5. The use according to claim 1, wherein said hematopoietic stem cells comprise CD34 and CD133 cells.
